(19) United States
(12) Patent Application Publication
(10) Pub. No.: US 2023/0134454 A1
(43) Pub. Date: May 4, 2023

(54) SEALED IMAGING DEVICES

(71) Applicant: Lumicell, Inc., Newton, MA (US)

(72) Inventors: Michael Bush, Arlington, MA (US); Joseph D'Anello, Braintree, MA (US); Steven Cappetta, Sudbury, MA (US); Jorge Ferrer, West Newton, MA (US); David Benjamin Strasfeld, Somerville, MA (US); Ling Xu, Belmont, CA (US); Sean Madden, Arlington, MA (US)

(73) Assignee: Lumicell, Inc., Newton, MA (US)

(21) Appl. No.: 17/977,179

(22) Filed: Oct. 31, 2022

Related U.S. Application Data
(60) Provisional application No. 63/275,855, filed on Nov. 4, 2021.

(51) Int. Cl. A61B 1/06

(52) U.S. Cl. CPC A61B 1/06; A61B 1/00096

(57) ABSTRACT

Embodiments related to sterilizable handheld medical imaging devices including a rigid imaging tip, a sealed housing, and/or a sealed cable assembly as well as their methods of use and manufacture are described.

### RELATED APPLICATIONS

This application claims the benefit of priority under 35 U.S.C. § 119(e) of U.S. Provisional Application Ser. No. 63/275,855, filed Nov. 4, 2021, the disclosure of which is incorporated herein by reference in its entirety.

### FIELD

Disclosed embodiments are related to sealed imaging devices and related methods of use.

### BACKGROUND

There are over one million cancer surgeries per year performed in the United States and nearly 40% of them miss resecting the entire tumor according to the National Cancer Institute Surveillance Epidemiology and End Results report. Residual cancer in the surgical bed is a leading risk factor for local tumor recurrence, reduced survival rates and increased likelihood of metastases. In a typical solid tumor resection, the surgeon removes the bulk of the tumor and sends it to pathology. The pathologist then samples the bulk tumor in a few locations and images a stained section under a microscope to determine if the surgeon has completely removed all of cancer cells from the patient. Should the pathologist find a portion of the stained sample with cancer cells bordering ink (a diagnostic known in the medical realm as “positive margin”), the surgeon may be instructed to resect more tissue. However, this pathology exercise is a time intensive procedure and often takes days for final results to be sent to the physician. Should a pathology report requiring additional resection return after the patient has completed the initial surgery, this may require the surgeon to perform a second surgery.

Some conventional surgical methods include employing fluorescent imaging devices. The imaging devices may employ one or more imaging agents configured to bind or otherwise be retained in cancerous or other abnormal tissue. The one or more imaging agents may fluoresce when exposed to an excitation light. In some cases, an imaging device may detect the presence of the fluorescent agent, thereby indicating the presence of additional cancerous or other abnormal tissue to remove during the surgical method.

### SUMMARY

In some aspects, sterilizable handheld medical imaging devices are provided.

In one embodiment, a sterilizable handheld medical imaging device comprises a housing, wherein an interior of the housing is sealed from a surrounding environment; a photosensitive detector disposed in the housing; a rigid imaging tip extending distally from the housing and optically coupled with the photosensitive detector; and a sealed cable assembly extending out from the housing, wherein the cable assembly is adapted and arranged to be selectively connected to an illumination source and wherein the cable assembly is configured be selectively connected to a computing device.

In another embodiment, a sterilizable handheld medical imaging device comprises a housing, wherein an interior of the housing is sealed from a surrounding environment; a photosensitive detector disposed in the housing; and a pressure inlet in fluidic communication with an interior of the housing.

In yet another embodiment, a sterilizable handheld medical imaging device comprises housing; a photosensitive detector disposed in the housing; a rigid imaging tip extending distally from the housing, wherein the rigid imaging tip comprises a proximal portion and a distal portion that is angled relative to the proximal portion, and wherein the rigid imaging tip includes a distal end portion defining a field of view of the imaging device; a dichroic mirror disposed between the rigid imaging tip and the photosensitive detector; and a mirror disposed at a junction between the proximal portion and the distal portion of the rigid imaging tip to optically couple the photosensitive detector to the distal end portion of the rigid imaging tip, wherein one or more of the interior surfaces of the housing and/or the rigid imaging tip comprise a biocompatible anodized material, wherein the biocompatible anodized material is configured to absorb light that deviates from an optical path extending through the imaging device.

In some aspects, a method of manufacturing an imaging device is provided.

In one embodiment, a method of manufacturing an imaging device comprises pressurizing an interior of a sealed housing of an imaging device; and monitoring a pressure drop within the sealed housing of the imaging device over a predetermined period of time.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic representation of a surgical bed being imaged with decreased magnification, according to some embodiments;

FIG. 2 is a perspective view of one embodiment of a sterilizable handheld medical imaging device, according to some embodiments;

FIG. 3 is a partially exploded view of one embodiment of a probe of a sterilizable handheld medical imaging device, according to some embodiments;

FIG. 4A is a side cross-sectional view taken along line 4A-4A of FIG. 3, according to some embodiments;

FIG. 4B is a perspective cross-sectional view taken along line 4A-4A of FIG. 3, according to some embodiments;

FIG. 5A is a schematic representation of a top view of the cable assembly of the sterilizable handheld medical imaging device of FIG. 2, according to some embodiments;

FIG. 5B is a top view of a portion of the cable assembly of FIG. 5A, according to some embodiments;

FIG. 5C is a cross-sectional view taken along line 5C-5C of FIG. 5B, according to some embodiments;

FIG. 5D is a side cross-sectional view taken along 5D-5D of FIG. 2, according to some embodiments;

FIG. 5E is a side cross-sectional view taken along 5E-5E of FIG. 2, according to some embodiments;

FIG. 6A-6B are perspective views of portions of the cable assembly of FIG. 2, according to some embodiments;

FIG. 7 is a flow chart illustrating a method of manufacturing a sterilization handheld medical device, according to some embodiments;

FIG. 8 is a schematic illustration of a lap joint, according to some embodiments.

### DETAILED DESCRIPTION

Handheld medical imaging devices have been developed for surgical operation to aid real time identification of tumor and removal thereof during surgery. Some handheld medical imaging devices may be employed to identify a tumor region based on the use of appropriate fluorescent imaging agents. The inventors have recognized the need to develop a sterilizable handheld medical fluorescent imaging device for surgical and/or other medical uses. For example, during operation, various portions of the imaging device may interact with and contact surgical areas in a subject. In order to prevent contamination of the surgical area, it may be desirable to sterilize the imaging device prior to use. However, conventional handheld medical fluorescence imaging devices typically include sensitive optical components in a separate cart mounted optics assembly that is not sterilized and/or the assemblies may be disposable such that the systems are not subjected to sterilization. Accordingly, the inventors have recognized that imaging devices including sensitive optical components and surfaces within portions of an imaging device that will be subjected to harsh sterilization treatments may be damaged. For example, during H₂O₂ plasma sterilization, various optical and/or semiconducting components contained within an imaging device may be corroded by the H₂O₂ plasma.

In addition to the above, the inventors have recognized a need for reducing the light leakage in various fluorescence imaging systems. For example, light leakage may be a problem associated with fluorescence and other imaging systems operating at relatively high illumination intensities. Such light leakage may result in reduced signal-to-noise ratio, lower imaging resolution, and may subsequently result in inaccurate identification of tumors and/or other abnormal tissue during use. Therefore, the inventors have recognized a need to reduce the presence of stray light leakage along the optical path of fluorescence and other types of imaging devices in which high intensity illumination (e.g., excitation) light may be used for imaging purposes.

In view of the above, the inventors have recognized the benefits associated with a sterilizable handheld medical imaging device having certain advantageous properties and constructions that prevent light leakage and/or impart the device with the ability to withstand sterilization. In some embodiments, a gas-tight imaging device comprising various sealed components, e.g., a sealed imaging tip, sealed device body, and/or sealed cable assembly, is disclosed herein. The sealed imaging device may advantageously provide a particular set of properties that allows the device to withstand sterilization. Such properties may include sterilizable surface coatings, tight tolerances between various components, and/or gas-tight seals at various joints, seams, and/or passthroughs in the device. These and other features may either be used separately and/or in combination with one another to provide the desired sterilizable sealed imaging device including optical components disposed within the sterilizable portion of the imaging device which may be damaged by the sterilization process if exposed to the surrounding environment. In some cases, the sealed imaging device may comprise additional components, e.g., such as a built-in pressure unit, that can be employed to test whether the device is properly sealed. The sealed imaging device may advantageously include various light absorbing surfaces (e.g., anodized surfaces) disposed on one or more internal surfaces of the imaging device along an optical path of the imaging device that may help to reduce the presence of stray light and minimize light leakage in the device.

In some embodiments, a sterilizable handheld medical imaging device and related method of manufacturing are disclosed herein. A sterilizable handheld medical imaging device, according to some embodiments, is a handheld medical imaging device that is capable of withstanding a number of sterilization cycles without being damaged and/or losing its functionalities. The handheld medical imaging device may be capable of withstanding various types of sterilization gas. In some cases, the sterilization gas comprises H₂O₂ plasma. It should be noted that any appropriate sterilization gas may be employed as the currently disclosure is not so limited.

In some embodiments, a sterilizable handheld medical imaging device is a fluorescence imaging device. The fluorescence imaging device, as described in more detail below, may be configured to provide an excitation light at a desired wavelength range that excites fluorescence of a matter (e.g., an imaging agent) and subsequently image the matter based on the emitted fluorescence. However, other types of imaging devices may employ the various constructions described herein including, but not limited to, time-resolved fluorescence, Raman spectroscopy, phosphorescence, and/or any other appropriate type of medical imaging system where it may be desirable to protect the optical components contained within a sterilizable portion of the device and/or to reduce the occurrence of stray light and/or light leakage within the device.

The sterilizable handheld imaging device may be employed in any of a variety of applications. According to exemplary embodiments described herein, a handheld medical imaging device may be employed to detect the presence of abnormal tissue with an appropriate imaging agent. In some embodiments, the medical imaging device may provide sufficient illumination of an excitation wavelength of the imaging agent to generate a fluorescence signal from the imaging agent that exceeds instrument noise of the imaging device. In some embodiments, the illumination provided by the medical imaging device may also result in an autofluorescence signal from healthy tissue. The medical imaging device may also detect abnormal tissue at sizes ranging from centimeters to sizes on the order of 10 micrometers to tens of micrometers. Other size scales are also possible. As described in more detail below, in some embodiments, it may be desirable for the medical imaging device to be able to image a large field of view in real-time and/or be relatively insensitive to human motions inherent in a handheld device as well as natural motions of a patient involved in certain types of surgery such as breast cancer and lung cancer surgeries. The imaging device may either be used for imaging surgical beds, such as tumor beds, or it may be used for imaging already excised tissue as the disclosure is not so limited.

In some embodiments, the sterilizable handheld medical imaging device comprises a housing configured to house a body of the imaging device and associated components therein. In some cases, a plurality of optical components and electronic components may be disposed within the housing of the device body. For example, in one set of embodiments, a photosensitive detector is disposed in the housing of the device body. Additional components that may be disposed within the housing of the body include, but are not limited to, a light source, light guides (e.g., fiber optic cables), light directing elements (e.g., mirrors), one or more filters, one or more lenses, optical and/or detector connectors, combinations of the forgoing, and/or any other appropriate component. Each of the above-referenced components is described in more detail below.

In some embodiments, an interior of the housing is sealed from a surrounding environment. That is, the interior volume of the housing including one or more components (e.g., optical and electrical components of the device body) disposed therein may not be in fluidic communication with the surrounding environment. In some cases, such a housing may advantageously protect the interior of the device body from being exposed to caustic and/or corrosive sterilization gases (e.g., H₂O₂ plasma) in the surrounding environment. Accordingly, various interior components (e.g., the photosensitive detector, light source, light guide(s), light directing elements (e.g., mirrors), one or more filters, optical and/or detector connectors, etc.) disposed within the housing of the device body may be shielded from exposure to sterilization gases during sterilization.

In some embodiments, the sterilizable handheld medical imaging device further comprises a rigid imaging tip extending distally from the housing and optically coupled with the photosensitive detector. For example, in one embodiment, the medical imaging device may include a rigid imaging tip including a distal end defining a focal plane at a fixed distance from an optically associated photosensitive detector. For example, a distally extending member may define at its distal end a focal plane of the photosensitive detector. Depending on the embodiment, optics associated with the photosensitive detector may either fix a focus of the photosensitive detector at the focal plane located at the distal end of the rigid imaging tip, or they may permit a focus of the photosensitive detector to be shifted between the focal plane located at the distal end of the rigid imaging tip and another focal plane located beyond the distal end of the rigid imaging tip. While any appropriate photosensitive detector might be used, exemplary photosensitive detectors include a charge-coupled device (CCD) detector, a complementary metal-oxide semiconductor (CMOS) detector, and an avalanche photo diode (APD). The photosensitive detector may include a plurality of pixels such that an optical axis passes from the focal plane of the rigid imaging tip to the photosensitive detector.

In some embodiments, the rigid imaging tip comprises a proximal portion and a distal portion that is angled relative to the proximal portion. In some cases, the bend formed between the proximal and distal potion of the rigid imaging tip may facilitate access of a medical imaging device into a surgical site. Any appropriate angle between the proximal and distal portions to facilitate access to a desired surgical site might be used, as described in more detail below. For example, in some embodiments, the distal portion of the rigid imaging tip may be angled by at least about 25°, 30°, 35°, 40°, 45°, 50°, 55°, or 60° relative to the proximal portion. In some embodiments, the distal portion of the rigid imaging tip may be angled by no more than about 65°, 60°, 55°, 50°, 45°, 40°, 35°, or 30° relative to the proximal portion. Any of the above-reference ranges are possible (e.g., at least about 25° and no more than about 65°). Other ranges are also possible including imaging tips without an angled portion.

In some embodiments, the sterilizable handheld medical imaging device further comprises a sealed cable assembly extending out from the housing at a side opposite the rigid imaging tip. In some embodiments, the sealed cable assembly is adapted and arranged to be selectively connected to an illumination source and a computing device. For example, the cable assembly may function to connect the light source and the photosensitive detector to an external illumination source, a power source and/or processor, respectively. The sealed cable assembly may comprise a plurality of cables, including but not limited to optical cables, electrical cables, air lines, etc. For example, in one set of embodiments, the cable assembly comprises a hybrid cable comprising a fiber optic cable and an UCS cable. The sealed cable assembly may further comprise a plurality of components associated with the cables, including but not limited to, cable connectors, cable sheaths, etc.

A sealed cable assembly, according to some embodiments, is arranged and constructed such that the interior of the cable assembly is not in fluidic communication with a surrounding environment in at least one configuration. For example, the sealed cable assembly may have a substantially impervious or gas-tight structure such that the interior components (e.g., optical or electrical wires) of the cable assembly are protected from being exposed to a surrounding environment containing sterilization gases (e.g., H₂O₂ plasma). For example, the sealed cable assembly may comprise one or more protective coatings and/or layers encapsulating the plurality of cables and associated components as well as sealed connectors, caps configured to form a seal with one or more adjacent components, and/or any other appropriate construction to facilitate sealing the cable assembly relative to a surrounding environment.

In some embodiments, the sterilizable handheld medical imaging device further comprises a pressure unit coupled with the cable assembly. In some embodiments, the pressure unit comprises a pressure inlet associated with a portion of the cable assembly and a pressure conduit that extends from the pressure inlet into the interior of the housing. In other words, the pressure inlet may be in fluidic communication with an interior of the housing through the cable assembly. According to some embodiments, the pressure unit may be adapted and arranged to be connected to a separate pressure source in order to apply a positive pressure to the interior of the housing relative to the surrounding environment. The pressure may be applied to the housing interior from a pressure source such as a pump, a pressure regulated gas cylinder, or other pressure source connected to the pressure inlet and associated pressure conduit. As described in more detail below, such a pressure unit may advantageously be used to determine whether the imaging device has been properly sealed from a surrounding environment.

Depending on the embodiment, a medical imaging device can also include one or more light directing elements for selectively directing light from an illumination source comprising an excitation wavelength of an imaging agent towards a distal end of the device while permitting emitted light comprising an emission wavelength of the imaging agent to be transmitted to the photosensitive detector. In one aspect, a light directing element comprises a dichroic mirror positioned to reflect light below a wavelength cutoff towards a distal end of an associated imaging tip while permitting light emitted by the imaging agent with a wavelength above the wavelength cutoff to be transmitted to the photosensitive detector. However, it should be understood that other ways of directing light towards a distal end of the device might be used including, for example, fiber optics, LEDs located within the rigid tip, and other appropriate configurations.

In some embodiments, the light directing element comprises a dichroic mirror disposed between the rigid imaging tip and the photosensitive detector disposed in the housing. In some embodiments, the imaging device may include various additional light directing elements, such as a light source mirror configured to redirect light from an illumination source towards the dichroic mirror. In embodiments in which the rigid imaging tip comprises a bend at a junction between the proximal portion and the distal portion, a light directing element comprising a mirror may be disposed at the junction of the rigid imaging tip. As described in more detail below, the mirror at the junction of the imaging tip may be adapted to bend an optical path through the angled or bent rigid imaging tip.

An imaging device may also include appropriate optics to focus light emitted from within a field of view of the device onto a photosensitive detector with a desired resolution. To provide the desired resolution, the optics may focus the emitted light using any appropriate magnification onto a photosensitive detector including a plurality of pixels. Depending on a size of the individual pixels, the optics may either provide magnification, demagnification, or no magnification as the current disclosure is not so limited. Without wishing to be bound by theory, a typical cancer cell may be on the order of approximately 15 μm across. In some embodiments, an optical magnification of the optics within a medical imaging device may be selected such that a field of view of each pixel may be equal to or greater than about 1 μm, 2 μm, 3 μm, 4 μm, 5 μm, 10 μm, 15 μm, 30 μm, or any other desired size. Additionally, the field of view of each pixel may be less than about 100 μm, 50 μm, 40 μm, 30 μm, 20 μm, 10 μm, or any other desired size scale. In one specific embodiment, the field of view per pixel may be between about 5 μm and 100 μm inclusively. In another embodiment, the field of view per pixel may be between about 5 μm and 50 μm inclusively.

In embodiments, the medical imaging device may be associated with and/or coupled to one or more illumination sources. For example, a first illumination source may be adapted and arranged to provide light including a first range of wavelengths to a light directing element that reflects light below a threshold wavelength towards a distal end of a rigid imaging tip and transmits light above the threshold wavelength. However, other ways of directing light from the one or more illumination sources toward the distal end of the rigid imaging tip including fiber optics and LEDs located within the device or rigid imaging tip might also be used. Regardless of how the light is directed, the first range of wavelengths may be selected such that it is below the threshold wavelength and thus will be reflected towards the distal end of the rigid imaging tip to illuminate the device's field of view. The illumination source may either be a constant illumination source or a pulsed illumination source depending on the particular embodiment. Additionally, the first range of wavelengths may be selected such that it corresponds to an excitation wavelength of a desired imaging agent. It should be understood that the specific wavelength will be dependent upon the particular imaging agent, optics, as well as the sensitivity of the photosensitive detector being used. However, in one embodiment, the first range of wavelengths may be between or equal to about 300 nm to 1,000 nm, 590 nm to 680 nm, 600 nm to 650 nm, 620 nm to 640 nm, or any other appropriate range of wavelengths depending on the particular imaging agent being used. Additionally, the first illumination source may be adapted to provide between about 10 mW/cm² to 200 mW/cm² at a desired focal plane for imaging tissue within a surgical bed, though other illumination intensities might also be used. For example, a light intensity of 10 mW/cm² to 40 mW/cm², 10 mW/cm² to 60 mW/cm², 10 mW/cm² to 80 mW/cm², 10 mW/cm² to 100 mW/cm², 25 mW/cm² to 60 mW/cm², 25 mW/cm² to 80 mW/cm², 25 mW/cm² to 100 mW/cm², 50 mW/cm² to 200 mW/cm², 100 mW/cm² to 200 mW/cm², or 150 mW/cm² to 200 mW/cm² could also be used. Depending on the particular imaging agent being used, the various components of the medical imaging device may also be constructed and arranged to collect emission wavelengths from an imaging agent that are about 300 nm to 1,000 nm, 590 nm to 680 nm, 600 nm to 650 nm, 620 nm to 640 nm, or any other appropriate range of wavelengths.

An exemplary imaging agent capable of providing the desired detection depths noted above is pegulicianine (LUM015). Pegulicianine and its use is further described in U.S. Patent Application Publication No. 2011/0104071 and U.S. Patent Application Publication No. 2014/0301950, which are included herein by references in their entirety. Other appropriate fluorophores that might be included in an imaging agent include, but are not limited to, Cy3, Cy3.5, Cy5, Alexa 568, Alexa 546, Alexa 610, Alexa 647, ROX, TAMRA, Bodipy 576, Bodipy 581, Bodipy TR, Bodipy 630, VivoTag 645, and Texas Red. Of course, one of ordinary skill in the art will be able to select imaging agents with fluorophores suitable for a particular application.

While various combinations of optical components and illumination sources are described above and in reference to the figures below, it should be understood that the various optical components such as filters, dichroic mirrors, fiber optics, mirrors, prisms, and other components are not limited to being used with only the embodiments they are described in reference to. Instead, these optical components may be used in any combination with any one of the embodiments described herein.

In some embodiments, the sterilizable handheld medical imaging device may include certain features and/or constructions that impart the device with the capability to withstand sterilization. For example, as noted above, the electrical and/or optical components within the interior of various portions of the imaging device (e.g., cable assembly, device body, etc.) may be encapsulated by housings, temporary coverings, protective coatings and/or layers such that the interior components with the device are sealed from a surrounding environment during sterilization.

Additionally or alternatively, the imaging device may comprise exterior surfaces that are resistant to sterilization. In some cases, at least a portion of the external surfaces of the imaging device may comprise a material that is resistant to sterilization gases. For example, in one embodiment, the external surfaces of the sealed cable assembly may comprise a polymeric material resistant to sterilization gases. Non-limiting examples of such materials include polypropylene (PP), stainless steel (SS), polycarbonate (PC), polyurethanes (PU), polyvinyl chloride (PVC), thermoplastic elastomer (TPE), thermoplastic natural rubber (TPNR), thermoplastic epoxidized natural rubber (TPENR), thermoplastic vulcanizate (TPV) (e.g., Santoprene™), and/or silicone. In one embodiment, a substantial percentage (e.g., at least 50%, at least 75%, at least 90%, at least 95%, at least 98%, or all) of the external surfaces of the sealed cable assembly comprises a thermoplastic elastomer (e.g., TPV, TPNR, TPENR, etc.). In some such embodiments, the thermoplastic elastomer comprises a blend of polymers, e.g., such as vulcanized ethylene propylene diene monomer (EPDM) rubber in a thermoplastic matrix of polypropylene (PP). In some embodiments, as described in more detail below, various sterilization resistant adhesives (e.g., epoxies, UV curable adhesives, etc.) may be applied to various regions (e.g., seams, joints, external surfaces etc.) of the imaging device (e.g., the housing and/or the rigid imaging tip) to facilitate bonding, potting, and layer-coating of various components of the device. Additionally, in some embodiments, at least a portion of the external surfaces associated with the rigid imaging tip and/or the housing of the device body may be anodized. For example, in some cases, the rigid imaging tip and/or the housing of the device body may include anodized exterior surfaces that are resistant to sterilization. Alternatively or additionally, as described in more detail below, at least a portion of the interior surfaces associated with the rigid imaging tip and/or the housing may be anodized. The anodized external surfaces may have any of a variety of properties described elsewhere herein with respect to the anodized interior surfaces.

In some embodiments, the imaging device may be assembled from individual pieces, such as the rigid imaging tip, the sealed housing, the sealed cable assembly. In order to form a sterilizable imaging device that is gas-tight, proper seals between the joints, seams, and/or pass throughs may be desirable. For example, the imaging device comprises various types of sealed joints, seams, pass throughs, etc. A plurality of adhesive sealants (e.g., sterilization resistant adhesive sealants), gaskets, and/or other features may be employed to achieve proper seals between the joints, seams, and/or pass throughs, as described in more detail below.

In one set of embodiments, sealed lap joints may be employed for creating seals between various components. For example, in one embodiment, the housing of the device may be formed from two pieces of material (e.g., metal) joined together via a lap joint. In some cases, it may be desirable to seal the lap joint via at least two or more seals. For instance, a first adhesive may be employed to form a first seal. To form the first seal, a first adhesive (e.g., a structural adhesive such as epoxy) may be applied to an external perimeter of a lap joint formed from two pieces of materials. Alternatively, to form the first seal, a first adhesive (e.g., a structural adhesive) may be applied to an inner edge of each of the two pieces of material. The two pieces of materials may then be joined together at the corresponding inner edges to form a sealed lap joint. In some instances, the first adhesive may be cured to form the first seal. In some embodiments, a second adhesive may be applied to a portion of the lap joint to form a second seal on the lap joint. In some embodiments, the second adhesive may be applied to an outer surface of the lap joint to reinforce and seal the joint. The second adhesive may comprise a light (e.g., UV) curable material in some embodiments. It should be noted that the imaging device may include other types of joints including either a single or multi-layer seal as detailed above as the present disclosure is not so limited. It should also be noted that the method described above (e.g., formation of two seals, etc.) may be employed to seal any appropriate type of joints in the imaging device. In some cases, the adhesives may be biocompatible and sterilization resistant (e.g., resistant to H₂O₂ plasma). Non-limiting examples of adhesives include light curable adhesives, heat curable adhesives (e.g., one-part adhesives, cyanoacrylate, etc.), epoxies (e.g., one-part or two-part epoxies, bisphenol A diglycidyl ether resin, EpoTek® MED-320, EpoTek® MED-353ND, etc.), etc. Non-limiting examples of appropriate adhesive chemistries include cyanoacrylates, bisphenols, novolaks, aliphatics, halogenated, and glycidylamines, etc. Non-limiting examples of UV curable adhesives include acrylated polyesters, acrylated urethanes (e.g., UV Cure Dymax® 1405), acrylated silicones, etc. Other types of biocompatible and sterilization resistant adhesives may also be used, as the present disclosure is not so limited.

In some embodiments, an imaging device comprises various sealed pass throughs. In some instances, a seal plug may be employed to seal the various pass throughs. For example, in one embodiment, the imaging device includes a tapered housing portion that is configured to compress and seal any cable(s) entering the housing of the device. The seal plug may be sized such that its inner diameter is substantially matched to, compresses, or otherwise forms a desired fit with the outer diameter of the sealed cable(s). In some instances, a sealant such as a structural adhesive may be applied to the outer surface of the cable(s) and the inner surface of the pass through to further seal the cable pass through.

In some embodiments, a sterilizable handheld medical imaging device may be configured to withstand a relatively high number of sterilization cycles. For example, in some embodiments, the sterilizable handheld medical imaging device is capable of withstanding at least 3, 5, 10, 15, 20, 25, 30, 40, 60, 80, 100, 120, 140, 160, 180, and/or any appropriate number of sterilization cycles. In some embodiments, the sterilizable handheld medical imaging device is capable of withstanding up to 20, 25, 30, 40, 60, 80, 100, 120, 140, 160, 180, 200, 250, 300, and/or any appropriate number of sterilization cycles. Combinations of the above-referenced ranges are also possible (e.g., at least 20 and up to 200 sterilization cycles, at least 20 and up to 100 sterilization cycles, or other combination). Other ranges are also possible.

The sterilization cycles may be carried out using any of a variety of temperature and/or pressures. For examples, in some cases, a relatively low temperature and/or pressure may be employed during the sterilization cycles. In some embodiments, the temperature may be at least 20° C., 30° C., 40° C., 50° C., 60° C., 70° C., 80° C., and/or any appropriate temperature. In some embodiments, the temperature may be no more than 100° C., 80° C., 70° C., 60° C., 50° C., 40° C., 30° C., and/or any appropriate temperature. Combinations of the above-referenced ranges are also possible (e.g., at least 40° C. and up to 60° C., at least 20° C. and up to 100° C.). Other ranges are also possible. In some embodiments, the applied pressure may be at least 10 kPa, 50 kPa, 100 kPa, and/or any appropriate pressure. In some embodiments, the applied pressure may be no more than 150 kPa, 100 kPa, 50 kPa, and/or any appropriate pressure. Combinations of the above-referenced ranges are also possible (e.g., at least 10 kPa and no more than 100 kPa). Other ranges are also possible.

In some embodiments, a sterilizable handheld medical imaging device may include a plurality of anodized surfaces. For example, it may be desirable to anodize at least a portion of interior surfaces of the imaging device such that stray light (e.g., light that deviates from an optical path) can be absorbed by the interior anodized surfaces. The anodized interior surfaces may advantageously reduce light leakage into the surrounding environment, thereby leading to increased imaging resolution and reduced noise levels.

In some embodiments, the sterilizable handheld medical imaging device may include a plurality of anodized interior surfaces. The anodized interior surfaces may include any interior surfaces associated with various non-optical components within the device. Non-optical interior surfaces, according to some embodiments, may refer to interior surfaces that are not positioned directly in an optical path extending through the imaging device. Conversely, it should be noted that any optical components that are positioned in the optical path extending through the imaging device lack anodized surfaces. In other words, surfaces associated with optical components that are involved in generating, transmitting, and/or receiving light along the optical path are not anodized. Example of such optical components include light directing elements (e.g., a dichroic mirrors, mirrors, prisms, etc.), light source (e.g., fiber optics), lenses, apertures, etc.

According to some embodiments, the optical path comprises an illumination path and an imaging path. For example, in one embodiment, the illumination path is a light path that originates from an illumination source (e.g., external illumination source), travels via an optical cable (e.g., fiber optics cable) within the cable assembly into the housing of the device body, reflects off the dichroic mirror disposed between the rigid imaging tip and the photosensitive detector, and further reflects off the mirror disposed at the junction between the proximal portion and the distal portion of the rigid imaging tip before exiting the distal end the rigid distal tip. In some embodiments, an imaging path refers to a light path that originates at the distal end of the imaging tip, reflects off the mirror disposed at the junction between the proximal portion and distal portion of the rigid imaging tip, and proceeds through the dichroic mirror to the photosensitive detector in the housing. In some embodiments, a portion of the illumination path and a portion of imaging path are coincident along a length of the imaging device between the dichroic mirror and the distal end of the imaging device. It should be noted that any suitable illumination and/or imaging path may be employed in a medical imaging device, as the present disclosure is not so limited.

In some embodiments, at least a portion of the housing and/or the rigid imaging tip comprises one or more anodized interior surfaces. In some such embodiments, the one or more anodized interior surfaces may be configured to absorb light that deviates from the optical path (e.g., illumination and/or imaging path) extending through the imaging device. In some embodiments, the anodized interior surfaces may have a certain set of desirable light absorption properties. For example, the anodized interior surfaces may be capable of selectively absorbing stray light having a wavelength corresponding to the emission or excitation wavelength ranges of a desired imaging agent. For example, as noted above, a light having a first wavelength (e.g., an excitation wavelength) may travel along a first optical path (e.g., the illumination path) to excite the imaging agent. The imaging agent, upon excitation, may emit light at a second wavelength (e.g., an emission wavelength) along a second optical path (e.g., the imaging path). In some cases, the anodized interior surfaces may be employed to absorb any stray light that deviates from the optical path having the first wavelength and/or second wavelength.

For example, in embodiments in which LUM015 is used at the imaging agent, the anodized interior surfaces may be configured to absorb light having an emission wavelength of about 650 nm and an excitation wavelength of about 680 nm. The anodized interior surfaces may be configured to absorb light at wavelengths corresponding to the excitation and emission ranges described herein for various imaging agents.

In some embodiments, the anodized interior surfaces may have a coating and/or color that impart the surfaces with the desired absorptive properties. For example, in some embodiments, the anodized interior surfaces may be inherently absorptive in a desired range of wavelengths and/or the anodized surface may incorporate a dye having the desired absorptive properties. Alternatively, a separate coating may be disposed on a surface to provide the desired absorptive properties. In one embodiment, a black dye may be used in the formation of black anodized interior surfaces of the device. Other colors of dye may also be used, as long as the anodized interior surfaces are capable of absorbing a substantial amount of the deviated light. For example, the anodized interior surfaces may be capable of absorbing at least 50% (e.g., 60%, 70%, or any other appropriate percentage) of all deviated light and/or deviated light having a particular range of wavelengths.

The anodized interior surfaces may exhibit any appropriate range of surface roughnesses. In some embodiments, the interior anodized surfaces may have an average surface roughness (measured as a root-mean square (RMS) value) of at least 1 micro-inch, 2 micro-inches, 4 micro-inches, 8 micro-inches, 16 micro-inches, 32 micro-inches, 63 micro-inches, 125 micro-inches, 250 micro-inches, 500 micro-inches, 1000 micro-inches, and/or any RMS appropriate values. In some embodiments, the interior anodized surfaces have an average surface roughness (measured as a root-mean square (RMS) value) of no more than 2000 micro-inches, 1000 micro-inches, 500 micro-inches, 250 micro-inches, 125 micro-inches, 63 micro-inches, 32 micro-inches, 16 micro-inches, 8 micro-inches, 4 micro-inches, 2 micro-inches, and/or a RMS appropriate values. Combination of the above-referenced ranges are possible (e.g., at least 1 micro-inch and no more than 2000 micro-inches). In the above ranges, an inch is equal to 0.0254 inches. Other ranges are also possible. The RMS average may be determined by measuring an average of height deviations of microscopic peaks and valleys from a mean value according to descriptions provided in ASME B46.1 or any other appropriate measurement standard.

The anodized surfaces (e.g., interior and/or exterior surfaces) may have any of a variety of appropriate thicknesses. In some embodiments, the anodized surfaces have an average thickness of at least 30 μm, 35 μm, 40 μm, 45 μm, 50 μm, 60 μm, 70 μm, 80 μm, 100 μm, 125 μm, 150 μm, and/or any appropriate values. In some embodiments, the anodized surfaces have an average thickness of no more than 200 μm, 150 μm, 125 μm, 100 μm, 80 μm, 70 μm, 60 μm, 50 μm, 45 μm, 40 μm, 35 μm, and/or any appropriate values. Combination of the above-referenced ranges are possible (e.g., at least 30 μm and no more than 150 μm, at least 35 μm and no more than 80 μm, or at least 45 μm and no more than 60 μm, etc.). Other ranges are also possible.

The anodized surfaces (e.g., interior and/or exterior anodized surfaces) may comprises any appropriate materials. In some cases, the anodized surfaces comprises a biocompatible material. For example, in one set of embodiments, the anodized surfaces comprises a biocompatible anodized aluminum. Other anodized metals are also possible, such as titanium and alloys thereof, stainless steel, etc., as the present disclosure is not so limited.

Certain aspects of the present disclosure are directed to a method of manufacturing a sterilizable handheld medical imaging device described herein.

In some embodiments, upon assembly, the sterilizable handheld medical device includes a sealed housing, a rigid imaging tip extending distally from the housing, a sealed cable assembly extending out from the housing, and a selectively sealable pressure inlet associated with a portion of the cable assembly. The pressure inlet may be configured to be in fluid communication with the interior of the housing. As described below, certain aspects of the manufacturing relate to performing a pressure test on the device via the pressure inlet to determine whether the device has been properly sealed.

During manufacturing, an interior of the sealed housing of the imaging device may be pressurized by applying a positive pressure through the pressure inlet. In some embodiments, the applied positive pressure may be at least 25 kPa, 30 kPa, 35 kPa, and/or any appropriate value relative to an exterior pressure. In some embodiments, the applied positive pressure may be no more than 40 kPa, 35 kPa, 30 kPa and/or any appropriate values. Combinations of the above-reference values may be possible (e.g., at least 25 kPa and no more than 40 kPa). Other ranges are also possible.

In some embodiments, a pressure drop within the sealed housing of the imaging device may be monitored for a predetermined amount of time. In some embodiments, the pressure drop may be monitored for at least 5 minutes, 6 minutes, 8 minutes, 10 minutes, 15 minutes, an/or any appropriate period of time. In some embodiments, the pressure drop may be monitored for no more than 20 minutes, 15 minutes, 10 minutes, 8 minutes, 6 minutes, and/or any appropriate period of time. Combination of the above-referenced ranges are possible (e.g., at least 5 minutes and less than 20 minutes). Other ranges are also possible.

In some embodiments, the monitored pressure drop within the sealed housing may have a relatively low value. A relatively low pressure drop may indicate that the sealed housing has a relatively gas-tight and/or impervious structure and has been properly sealed from the surrounding environment. For example, in some cases, the pressure drop may be less than or equal to 10 kPa, 5 kPa, 1 kPa, 0.5 kPa, 0.1, kPa, and/or any appropriate value. In one specific embodiment, the observed pressure drop is less than 5 kPa. In some embodiments, no appreciable pressure drop is detected within the sealed housing during the predetermined amount of time.

In some embodiments, upon confirming that the housing is properly sealed, the imaging device may be subjected to at least one or more sterilization cycles via exposure to a sterilization gas (e.g., H₂O₂). Prior to sterilization, various inlets and/or openings into the interior of the imaging device may be sealed with a plug or cap. For example, in one embodiment, the associated pressure inlet may be sealed with either a detachable or permeant plug. For another example, a distal and/or proximal end of the one or more cables (e.g., electronic cables, optical cables) within the cable assembly may be sealed with cable caps.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

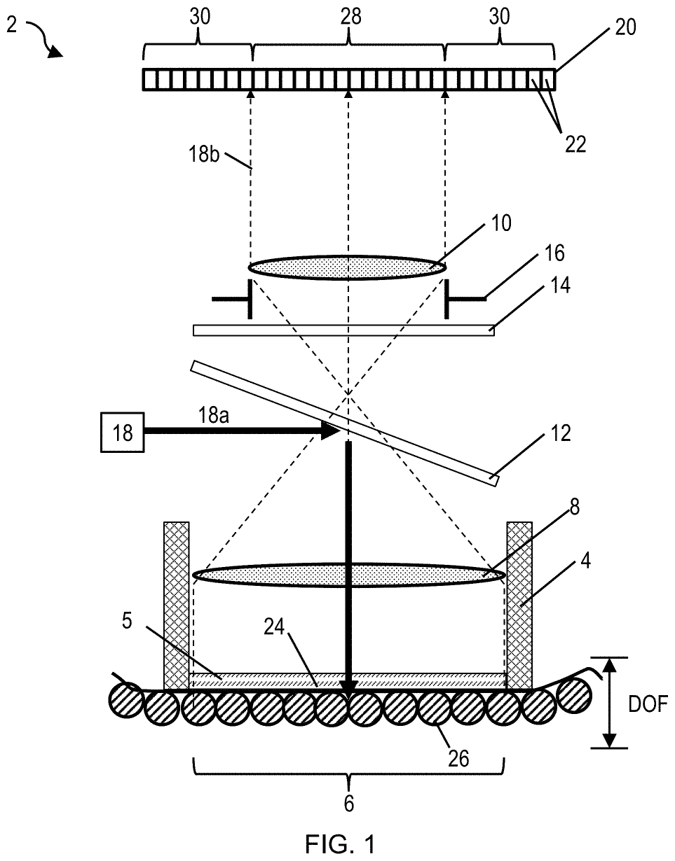

FIG. 1 depicts a schematic representation of exemplary embodiments for components of a medical imaging device 2. The medical imaging device may include a rigid imaging tip 4 at least partially defined by a distally extending member, frustoconical cylinder or other hollow structure. The rigid imaging tip 4 may be constructed and arranged to be held against tissue to fix a focal length of the medical imaging device relative to the tissue. As shown in FIG. 1, the rigid imaging tip includes an optically transparent window 5 that may be pressed into the tissue bed 24 to flatten the tissue at the fixed focal length of the medical imaging device. As depicted in FIG. 1, the rigid imaging tip 4 may also include an opening at a distal end that defines a field of view 6. The medical imaging device 2 may also include optics such as an objective lens 8, an imaging lens 10, and an aperture 16. The optics may focus light emitted from the field of view 6 onto a photosensitive detector 20 including a plurality of pixels 22. The medical imaging device may also include features such as a dichroic mirror 12 and a filter 14. While a doublet lens arrangement has been depicted in FIG. 1, it should be understood that other types of optics capable of focusing the field of view 6 onto the photosensitive detector 20 might also be used including, for example, fiber-optic bundles. Additionally, the photosensitive detector may correspond to any appropriate type of photosensitive detector configured to image or otherwise acquire a light-based signal from the field of view including photosensitive detectors such as a charge-coupled device (CCD), a complementary metal oxide semiconductor (CMOS) array, an avalanche photodiode (APD) array, or other appropriate detector.

As illustrated in FIG. 1, the medical imaging device may be positioned such that a distal end of the rigid imaging tip 4 may be pressed against a surgical bed 24 including one or more cells 26 which may be marked with a desired imaging agent. Instances where all, a portion, or none of the cells are marked with the imaging agent are contemplated. Pressing the rigid tip against the surgical bed may prevent out of plane and lateral tissue motion, which may allow for the use of collection optics with larger f numbers and consequently, larger collection efficiencies, smaller blur radii, and smaller depth of field. Additionally, pressing the rigid imaging tip 4 against the surgical bed may provide a fixed focal length between the tissue bed 24 and photosensitive detector 20. In some embodiments, the rigid imaging tip may have a length such that the distal end of the rigid imaging tip is also located at a focal plane of the photosensitive detector 20 in at least one mode of operation (e.g., when the photosensitive detector is focused on a fixed focal plane defined by the window 5). In some such embodiments, in at least one mode of operation the medical imaging device may have a fixed focal length between the tissue bed 24 and the photosensitive detector 20 as the tissue bed is pressed against the window 5. As shown in FIG. 1, the window 5 may be flat, such that the window flattens the tissue bed 24 into alignment with the distal end of the rigid imaging tip. In some embodiments, the medical imaging device may include a variable focus. According to such embodiments, in at least one mode of operation the focal plane may be adjustable, such that the focus may be set by a user based on the window 5 and tissue bed 24. For example, prior to use of the medical imaging device, the focal plane may be aligned with the window 5, or a position based at least in part on the window. As shown in FIG. 1, pressing the rigid imaging tip against the surgical bed may position the surgical bed 24 and the cells 26 contained therein within a predetermined distance (e.g., within a depth of field (DOF) of the imaging device) of the focal plane of the imaging device.

In some embodiments, it may be desirable to maintain a fixed distance between a distal end of the rigid imaging tip and the photosensitive detector. This may help to maintain the focus of tissue located within the focal plane defined by the distal end of the rigid imaging tip. Therefore, the rigid imaging tip may be adapted to resist deflection and/or deformation when pressed against a surgical bed such that tissue located within the focal plane defined by the distal end of the rigid imaging tip is maintained in focus.

During use, the medical imaging device may be associated with an illumination source 18 that directs light 18a with a first range of wavelengths towards the dichroic mirror 12. The first range of wavelengths may correspond to an excitation wavelength of a desired imaging agent. In some instances, the illumination source 18 may include appropriate components to collimate the light 18a. The illumination source 18 might also include one or more filters to provide a desired wavelength, or spectrum of wavelengths, while filtering out wavelengths like those detected by the photosensitive detector 20. In some embodiments, the dichroic mirror 12 may have a cutoff wavelength that is greater than the first range of wavelengths. Thus, the dichroic mirror 12 may reflect the incident light 18a towards a distal end of the rigid imaging tip 4 and onto the surgical bed 24. When the one or more cells 26 that are labeled with a desired imaging agent are exposed to the incident light 18a, they may generate a fluorescent signal 18b that is directed towards the photosensitive detector 20. The fluorescent signal may have a wavelength that is greater than the cutoff wavelength of the dichroic mirror 12. Therefore, the fluorescent signal 18b may pass through the dichroic mirror 12. The filter 14 may be a band pass filter adapted to filter out wavelengths other than the wavelength of the fluorescent signal. Alternatively, the filter 14 may permit other selected wavelengths to pass through as well. The fluorescent signal 18b may also pass through an aperture 16 to the imaging lens 10. The imaging lens 10 may focus the fluorescent signal 18b, which corresponds to light emitted from the entire field of view, onto a plurality of pixels 22 of the photosensitive detector 20. In some instances, the fluorescent signal 18b may be focused onto a first portion 28 of the photosensitive detector while second portions 30 of the photosensitive detector are not exposed to the fluorescent signal. However, in some embodiments, the fluorescent signal may be focused onto an entire surface of a photosensitive detector as the disclosure is not so limited.

Depending on the photosensitive detector used and the desired application, the one or more pixels 22 may have any desired size field of view. This may include field of views for individual pixels that are both smaller than and larger than a desired cell size. Consequently, a fluorescent signal 18b emitted from a surgical bed may be magnified or demagnified by the imaging device's optics to provide a desired field of view for each pixel 22, see demagnification in FIG. 1. Additionally, in some embodiments, the optics may provide no magnification to provide a desired field of view for each pixel 22.

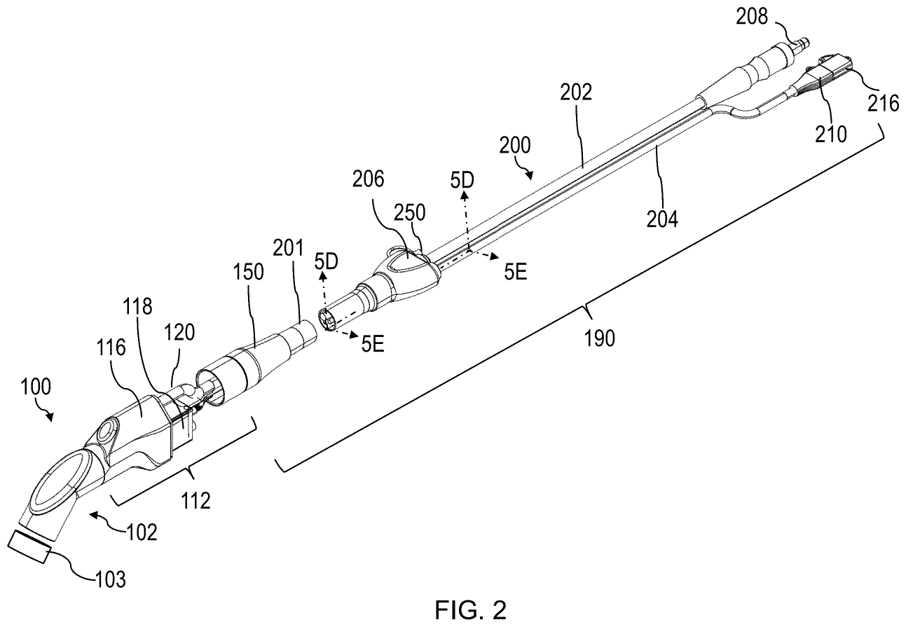

Having generally described embodiments related to a medical imaging device and an associated rigid imaging tip, specific embodiments of a medical imaging device and its components are described in more detail below with regards to FIGS. 2-5B.

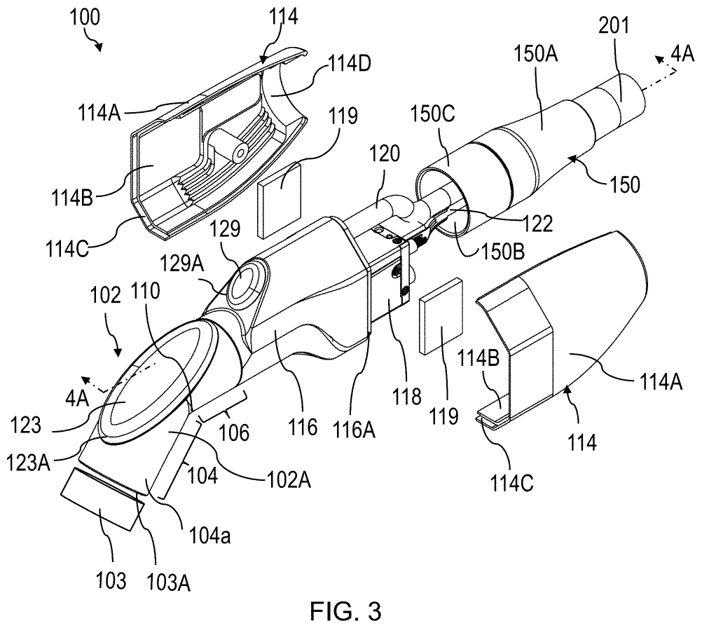

FIG. 2 depicts a perspective view of a sterilizable handheld medical imaging device 100. As shown, the imaging device 100 includes a body 112 with a housing 116. The imaging device includes a photosensitive detector 118 disposed within the housing 116, and a rigid imaging tip 102 extending distally from the housing 116 and optically associated with the photosensitive detector 118. The imaging device 100 may further comprise a sealed cable assembly 190 extending out from the housing 116. In some embodiments, the cable assembly may extend out from a portion of the housing and/or body opposite from the rigid imaging tip 102. It should be noted that FIG. 2 shows only a portion of the housing 116 for illustrative purposes. Thus, it should be understood that the housing 116 may include additional housing portions, such as a light source covering portion 114 arranged to house the photosensitive detector 118 as illustrated in FIG. 3 and described further below. As shown in FIG. 2, the cable assembly 200 comprises a hybrid cable 200. The cable assembly 200 may be adapted and arranged to selectively connect to an illumination source (not shown) and a computing device (not shown), as described in more detail below. As also described in more detail below, a pressure inlet 250 may be associated with the cable assembly 190 via a yoke 206 or other appropriate portion of the cable assembly. As noted previously, the pressure inlet may either be selectively sealable (i.e., may include a removeable seal such as a plug, openable valve, or other structure) or the pressure inlet may be permanently sealed after manufacture as the disclosure is not limited in this fashion.

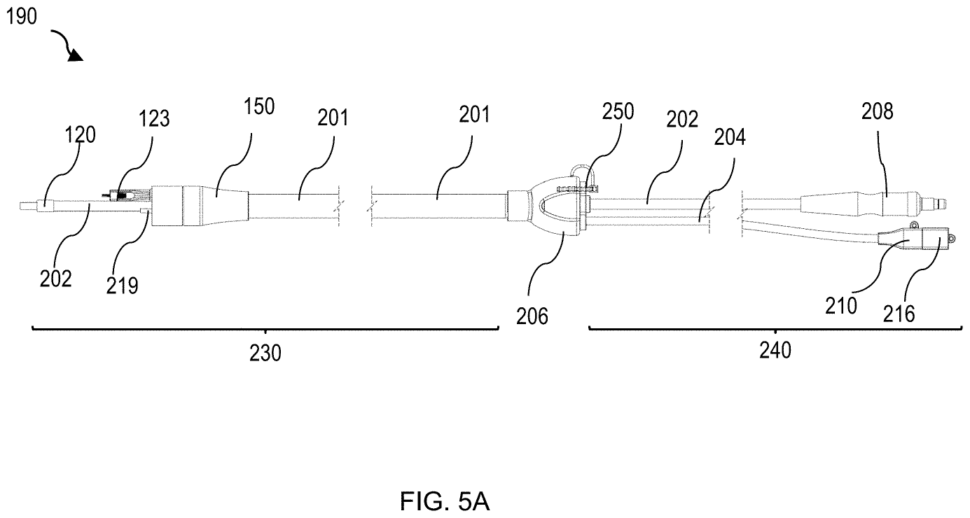

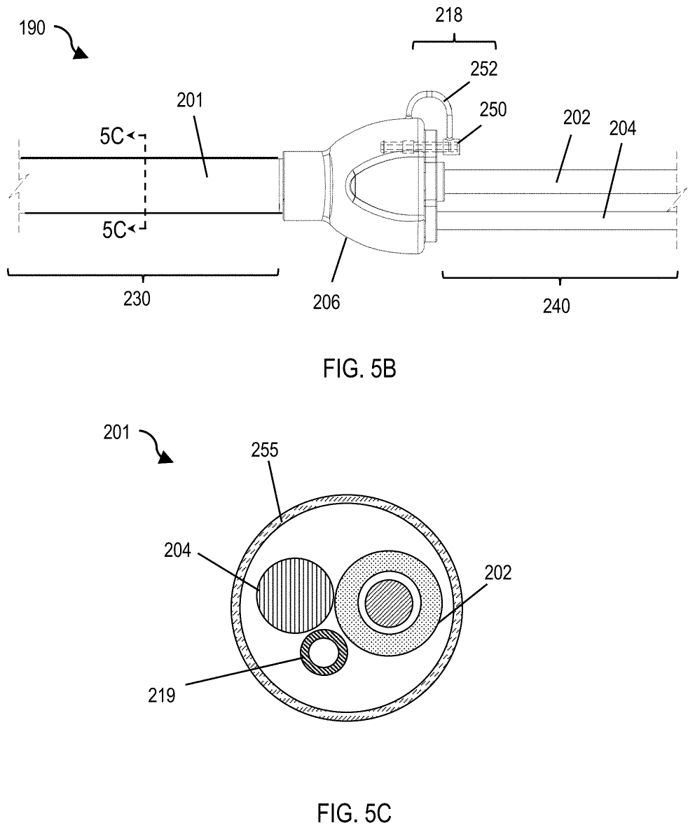

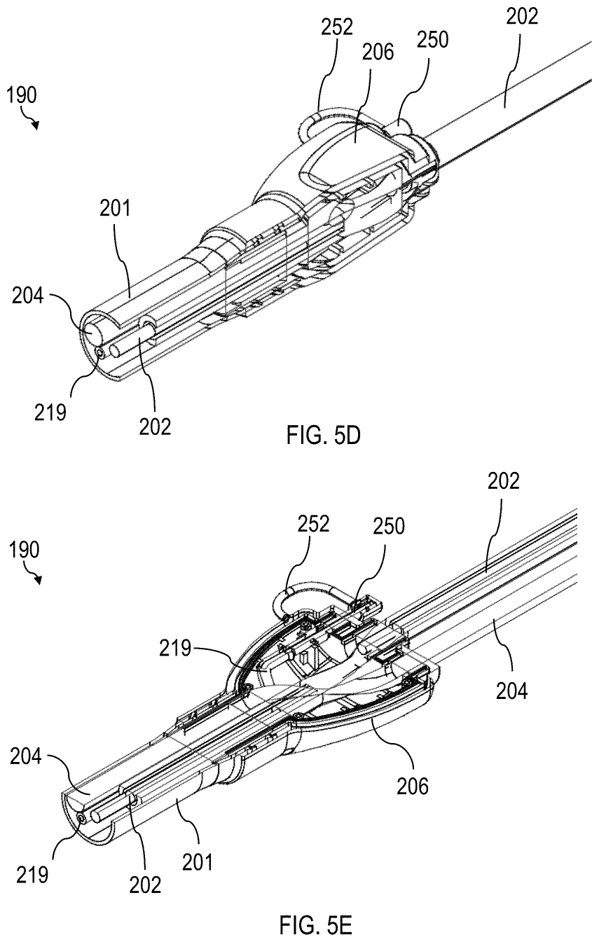

As shown in FIG. 2, the imaging device 100 includes a rigid imaging tip 102 configured to be placed on tissue to image the tissue at a focal length set by a distal end of the imaging tip. The imaging device includes a body 112 that may be manipulated by a user (e.g., a surgeon). In some embodiments as shown in FIG. 2, the body of the device includes a housing 116 having a portion that functions as a handle so that the device may be hand operated. The body houses a light source 120 and a photosensitive detector 118. The light source 120 may be configured to illuminate the targeted tissue for imaging. In particular, the light source 120 may be configured to provide an excitation light at a desired wavelength range that excites fluorescence of an imaging agent. As will be discussed further with reference to exemplary embodiments below, the light may pass from the light source 120 through several reflecting surfaces, lens, filters, and/or other optical elements before reaching the imaging tip 102. The light source 120 as shown in FIG. 2 is a fiber optic cable, which may be connected to an external illumination source via the hybrid cable 200. As shown in FIG. 2, the light source 120 and the photosensitive detector 118 are attached to a housing 116. The housing 116 may house the various optical components. The housing may also include the imaging tip 102. As shown in FIG. 2, the medical imaging device includes a removable tip 103 that may be attached to the imaging tip 102. As will be discussed further below, the removable tip 103 may include a window and may be configured to engage a tissue bed to flatten the tissue bed within a depth of field of the photosensitive detector 118. The housing 116 may also provide a handling surface (e.g., a handle) for a user of the medical imaging device 100. According to some embodiments as shown in FIG. 2, the medical imaging device may also include a tapered housing portion 150 which may assist in sealing the housing 116 from fluid ingress. In some embodiments, the tapered housing portion may compress and seal a portion 201 of the hybrid cable 200 entering the body 112. A portion 201 of hybrid cable 200 may be in the form of a monolithic cable bundle, as described in more detail below with respect to FIGS. 5A-5E.

According to the embodiment of FIG. 2, the medical imaging device 100 includes a hybrid cable 200. The hybrid cable may function to connect the light source 120 (e.g., a light guide such as the fiber optic cable depicted inside the imaging device) and the photosensitive detector 118 to an external illumination source, a power source and/or processor, respectively. As shown in FIG. 2, the hybrid cable includes an optical cable 202 configured to pipe light from an external illumination source to the light source 120. In some cases, the optical cable comprises fiber optics. The hybrid cable 200 may include a detector cable 204. In some embodiments, the detector cable 204 may transmit both power and imaging signals to and from the photosensitive detector to an associated power source and computing respectively in some embodiments. However, instances in which separate cables are used for power and signal transmission are also contemplated. Regardless of the specific arrangement, the detector cable 204 may connect the photosensitive detector 118 to a computing device including one or more processors configured to receive signals from the photosensitive detector. In some embodiments, the detector cable may employ a standardized protocol for data and power, such as USB 2.0, USB 3.0, USB-C, or any other suitable protocol. As sown in FIG. 2, the hybrid cable includes a yoke 206 which receives both the optical cable 202 and the detector cable 204. In some embodiments, the proximal cable is configured to provide a waterproof seal between the optical cable and the detector cable. The hybrid cable also includes an optical connector 208 configured to connect to an external illumination source. The hybrid cable also includes a detector connector 210 configured to connect the detector to an external device (e.g., a computing device). Of course, while a wired medical imaging device 100 is shown including a hybrid cable 200 in the embodiment of FIG. 2, in other embodiments data may be transmitted wirelessly to an external device (e.g., a computing device). For example, the medical imaging device 100 may include a wireless transmitter or transceiver configured to send or receive information from an external device (e.g., a computing device). In some embodiments, a medical imaging device 100 may be wired to an illumination source and power source but may transmit information wirelessly to an external device having one or more processors. Of course, any suitable combination of wired and wireless connections may be employed, as the present disclosure is not so limited.

FIG. 3 depicts a partially exploded view of a medical imaging device 100 including a distally extending rigid imaging tip 102. The rigid imaging tip 102 may include a distal portion 104 and a proximal portion 106. A distal end 104a of the rigid imaging tip located on the distal portion 104 may at least partly define a field of view for the imaging device. In some embodiments, the proximal portion 106 may be constructed to either be detachably or permanently connected to a housing 116 of the imaging device. In some embodiments, the rigid imaging tip may also be made from materials that are compatible with typical sterilization techniques such as various steam, heat, chemical, and radiation sterilization techniques.

As shown in FIG. 3, the medical imaging device 100 includes a removable tip 103 configured to be removably attached to the distal end 104a of the rigid imaging tip 102. The removable tip may be configured to protect the rigid imaging tip during use of the device with a tissue bed. In some embodiments, the removable tip 103 may include one or more optically transparent windows configured to allow light to pass through the removable tip. In some embodiments, the removable tip may be configured to be pressed against a tissue bed to flatten the tissue within a depth of field of a photosensitive detector 118. In some embodiments, the connection between the rigid imaging tip 102 and the removable tip 103 may include, for example, a snap on, screw on, suction, magnetic connection, and/or any other appropriate type of connection. This may provide multiple benefits including, for example, easily and quickly changing a rigid imaging tip during a surgical procedure as well as enabling the rigid imaging tip to be removed and sterilized. In some embodiments, the removable tip 103 may be removed from the medical imaging device after use.

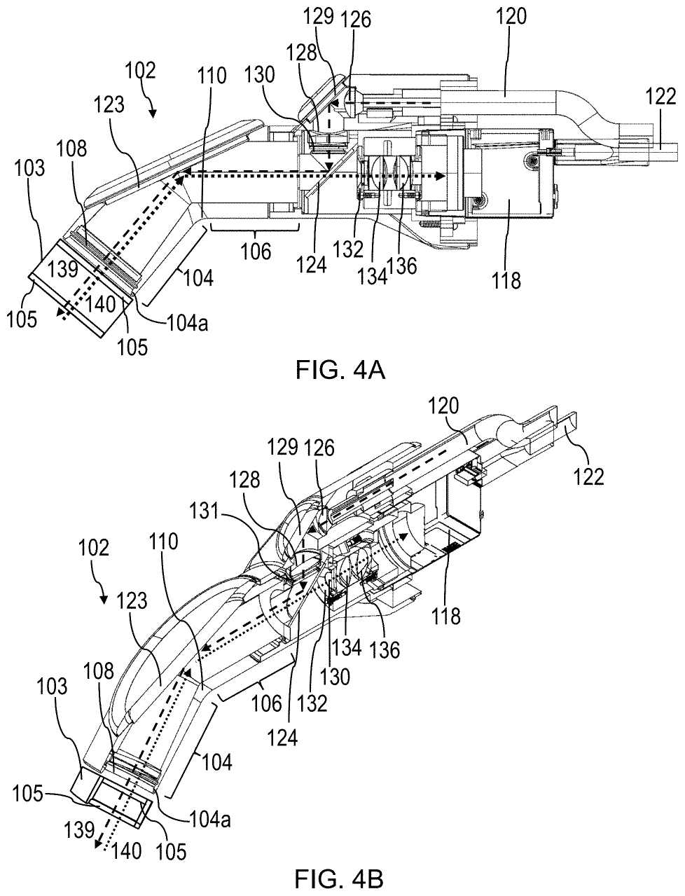

In some embodiments as shown in FIG. 3, the housing 116 of the medical imaging device 100 may include a light source covering portion 114. As noted above, an interior of the housing 116 may be sealed from a surrounding environment. The body 112 of the device may be sealed by housing 116 such that various internal components disposed within the housing 116 are sealed from the surrounding environment. For example, the various internal components nay include the photosensitive detector 118, the light source 120, the data output 122, various mirrors (e.g., dichroic mirror, mirror, etc.) as described in FIG. 4A-4B. The seals between the various portions of the housing, imaging tip, and cable pass through are detailed further below.

As shown in FIG. 3, the housing 116 is configured to mount the photosensitive detector 118 to the medical imaging device. The light source covering portion 114 houses thermal pads 119 configured to absorb heat from the photosensitive detector. In some embodiments, the light source covering portion 114 may be configured to cover the light source 120 and the photosensitive detector 118. In some embodiments, the photosensitive detector 118 may include an appropriate data output 122 for outputting data to an external device (e.g., a computing device). In some embodiments, the data output may include a detector cable, as described previously with reference to FIG. 2. Additionally, in some embodiments, the photosensitive detector may include a power input. In some embodiments, the power input may include a detector cable, as described previously with reference to FIG. 2. In some embodiments, the data output 122 may include an integrated power input to the photosensitive detector 118, for example, in the form of a detector cable (see FIG. 2, for example). In some embodiments, one or more light sources 120 associated with one or more separate illumination sources, not depicted, may be covered by the light source covering portion 114. As discussed previously, the light source 120 may provide light including at least a first range of excitation wavelengths to the medical imaging device 100. According to the embodiment of FIG. 3, the medical imaging device includes a tapered housing portion 150 configured to compress and seal any cable(s) entering the housing 116.

FIGS. 4A-4B depict cross sectional views of the medical imaging device of FIG. 3 taken along line 4A-4A. The cross sections of FIGS. 4A-4B depict the optical arrangement of the medical imaging device. As shown in FIGS. 4A-4B, the medical imaging device includes a rigid imaging tip 102 corresponding to a member distally extending from the housing 116 with an optically transparent or hollow interior. A distal end 104a of the rigid imaging tip 102 may define a focal plane located at a fixed distance relative to the optically coupled photosensitive detector 118 located on a proximal portion of the medical imaging device. In one embodiment, the optics coupling the rigid imaging tip and the photosensitive detector may include an objective lens 134 and an imaging lens 136 located between the rigid imaging tip and the photosensitive detector. The objective and imaging lenses 134 and 136 may focus light emitted from within a field of view of the rigid imaging tip onto a surface of the photosensitive detector 118 including a plurality of pixels. A magnification or demagnification provided by the combined objective and imaging lenses 134 and 136 may be selected to provide a desired field of view for each pixel.

As shown in FIGS. 4A-4B, the medical imaging device 100 may also include one or more dichroic mirrors 124 located between the photosensitive detector 118 and a distal end 104a of the rigid imaging tip. The dichroic mirror 124 may be adapted to reflect light below a cutoff wavelength towards the distal end of the rigid imaging tip and transmit light above the cutoff wavelength towards the photosensitive detector 118. In the current embodiment, the cutoff wavelength may be greater than an excitation wavelength of a desired imaging agent and less than an emission wavelength of the imaging agent. While any appropriate structure might be used for the dichroic mirror, in one embodiment, the medical imaging device includes a single dichroic mirror along an optical path of the medical imaging device.

In some embodiments as shown in FIGS. 4A-4B, the medical imaging device 100 may include one or more filters 130 located between the dichroic mirror 124 and the photosensitive detector 118. The one or more filters 130 may be adapted to permit light emitted from an imaging agent to pass onto the photosensitive detector while blocking light corresponding to excitation wavelengths of the imaging agent. Depending on the embodiment, the one or more filters may either permit a broad spectrum of wavelengths to pass or they may only permit the desired excitation wavelength, or a narrow band surrounding that wavelength, to pass as the disclosure is not so limited.

In some embodiments as shown in FIG. 4A-4B, an aperture stop 132 including an appropriately sized aperture may also be located between the rigid imaging tip 102 and the photosensitive detector 118. More specifically, the aperture stop 132 may be located between the dichroic mirror 124 and the imaging lens 136. Depending on the embodiment, the aperture may have an aperture diameter selected to provide a desired f number, depth of field, and/or reduction in lens aberrations. Appropriate aperture diameters may range from about 5 mm to 15 mm inclusively which may provide an image side f number between about 3 to 3.5 inclusively. However, other appropriate aperture diameters and f numbers are also contemplated.

During use of the medical imaging device 100, the light source 120 may receive light from an associated illumination source. The light source 120 may be any appropriate structure including, for example, fiber-optic cables used to transmit light from the associated illumination source to the medical imaging device. According to the embodiment of FIGS. 4A-4B, the light source 120 is configured to extend in a direction that is parallel to a longitudinal axis of a portion of the medical imaging device the light source extends through. Accordingly, as shown in FIGS. 4A-4B, the light source 120 is orientated parallel to the direction of imaging of the photosensitive detector 118 along an associated portion of the optical path though other orientations of these components may also be used as the disclosure is not so limited. In some embodiments, the light source 120 may be associated with optics such as an aspheric lens 126 disposed on a distal end of the depicted optical fiber bundle of the light source 120 to help collimate light directed towards the dichroic mirror 124. As shown in FIGS. 4A-4B, the light source may also include an additional collimating lens to further collimate light toward the dichroic mirror 124. The light source 120 may also be optically coupled with one or more filters 131 disposed between the light source and the dichroic mirror in order to provide a desired wavelength, or a spectrum of wavelengths, to the dichroic mirror 124 and ultimately the rigid imaging tip 102. This wavelength, or spectrum of wavelengths, may correspond to one or more excitation wavelengths of a desired imaging agent used to mark abnormal tissue for imaging purposes. Depending on the embodiment, the light source 120 may either be associated with a single illumination source, or it may be associated with multiple illumination sources. Alternatively, multiple light inputs may be coupled to the medical imaging device to provide connections to multiple illumination sources as the current disclosure is not so limited.

According to the embodiment of FIGS. 4A-4B, as the light source 120 is oriented parallel to a longitudinal axis of the medical imaging device, the dichroic mirror 124 is not in a direct optical path of the light source. Accordingly, as shown in FIGS. 4A-4B, the medical imaging device may include a light source mirror 129 configured to redirect the light from the light source 120 towards the dichroic mirror 124. That is, the light source mirror 129 reflects the light from the light source approximately 90 degrees toward the dichroic mirror 124. In some embodiments as shown in FIGS. 4A-4B, the light source mirror is disposed between the aspheric lens 126 and the collimating lens 128, though other arrangements are contemplated, and the disclosure is not so limited. The path of light provided by the light source is shown by light source path 139 (i.e., an illumination path), which is discussed further below. While a mirror is employed in the embodiment of FIGS. 4A-4B, in other embodiments other light bending elements may be employed, including, but not limited to, prisms, fiber optics, etc., as the present disclosure is not so limited.

It should be understood that the above components may be provided in any desired arrangement. Additionally, a medical imaging device may only include some of the above noted components and/or it may include additional components. However, regardless of the specific features included, an optical path 140 (i.e., an imaging path) of a medical imaging device may pass from a distal end 104a of a rigid imaging tip 102 to a photosensitive detector 118. For example, light emitted from within a field of view may travel along an optical path 140 (i.e., an imaging path) passing through the distal end 104a as well as the distal and proximal portions 104 and 106 of the rigid imaging tip. The optical path may also pass through the housing 116 including various optics to the photosensitive detector 118.

According to the embodiment of FIGS. 4A-4B, a medical imaging device 100 includes a rigid imaging tip 102 with a distal portion 104 and a proximal portion 106. The distal portion 104 may include a distal end 104a including an opening optically coupled with a photosensitive detector 118. The rigid imaging tip includes a window 108 integrated with the distal end 104a of the rigid imaging tip. The window 108 may be transparent to both the excitation wavelengths provided by an associated illumination source as well as wavelengths emitted from a desired imaging agent. While any appropriate shape might be used depending on the particular optics and algorithms used, in one embodiment, the window 108 may have a flat shape to facilitate placing tissue at a desired focal plane when it is pressed against a surgical bed. Additionally, as shown in the embodiment of FIGS. 4A-4B, the medical imaging device 100 includes a removable tip 103 configured to be removably attached to the distal end 104a of the rigid imaging tip 102. The removable tip may be configured to protect the rigid imaging tip during use of the device with a tissue bed. The removable tip 103 includes two optically transparent windows 105 configured to allow light to pass through the removable tip. In particular, the windows 105 may be transparent to both the excitation wavelengths provided by an associated illumination source as well as wavelengths emitted from a desired imaging agent. Of course, while two windows are shown in the embodiment of FIGS. 4A-4B, in other embodiments any suitable number of windows may be employed, as the present disclosure is not so limited. In some embodiments, the removable tip 103 may be configured to be pressed against a tissue bed to flatten the tissue within a depth of field of the photosensitive detector 118. For example, one of the windows 105 may be pressed against the tissue to flatten the tissue against the window. In some embodiments a focal plane of the photosensitive detector may be aligned with a distal window 105 of the removable tip 103, such that tissue pressed against the distal window is within a depth of field of the photosensitive detector. In some embodiments, the connection between the rigid imaging tip 102 and the removable tip 103 may include, for example, a snap on, screw on, suction, magnetic connection, and/or any other appropriate type of connection.

In some embodiments as shown in FIGS. 4A-4B, the rigid imaging tip 102 includes a bend 110 to facilitate access of a medical imaging device into a surgical site. For example, a distal portion 104 of the rigid imaging tip may be angled relative to a proximal portion 106 of the rigid imaging tip. Any appropriate angle between the proximal and distal portions to facilitate access to a desired surgical site might be used. However, in one embodiment, an angle between the proximal and distal portions may be between about 25° to 65°. For example, a rigid imaging tip may have an angle that is equal to about 45°. In embodiments including an angled distal portion, the rigid imaging tip 102 includes a mirror 123 adapted to bend an optical path 140 (i.e., an imaging path) and light source path 139 (i.e., an illumination path) through the bent rigid imaging tip. The mirror may be positioned at the bend 110 at a junction between the proximal portion and the distal portion of the rigid imaging tip, such that light traveling through the proximal portion 106 is reflected through the distal portion 104. Likewise, light traveling through the distal portion 104 is reflected by the mirror through the proximal portion 106. In this manner the mirror provides a reflective surface allowing for the transmission of both excitation light and light emitted from a desired imaging agent to travel through the rigid imaging tip 102. It should be understood that even though a bent configuration with a mirror 123 is shown in the exemplary embodiment of FIGS. 4A-4B, one or more other light bending components (e.g., prisms, fiber optics, etc.) may be employed, as the present disclosure is not so limited. Additionally, in some embodiments, a straight imaging tip may be employed without any mirror, as the present disclosure is not so limited.

According to the embodiment of FIGS. 4A-4B, the light source path 139 (i.e., an illumination path) and optical path 140 (i.e., an imaging path) are substantially parallel along at least a portion of a length of the imaging device. The optical path 140 originates at the distal end 104a, reflects off the mirror 123 and proceeds through the dichroic mirror 124 to the photosensitive detector. The light source path originates at the light source 120, reflects off the light source mirror 129, reflects off the dichroic mirror 124 toward the rigid imaging tip 102, and finally reflects off the mirror 123 and exits the distal end 104a of the rigid distal tip. Accordingly, the light source path 139 and optical path 140 are parallel from the dichroic mirror 124 through the distal end 104a of the rigid imaging tip. In some embodiments, a portion of the optical path 140 and a portion of the light source path 139 are coincident along a length of the imaging device between the dichroic mirror 124 and the distal end 104a. Of course, any suitable optical path and light source path may be employed in a medical imaging device, as the present disclosure is not so limited.

FIG. 5A depicts a schematic representation of a top view of the sealed cable assembly 190 in the handheld medical imaging device 100 of FIG. 2. As shown in FIG. 5A, the cable assembly 190 includes a proximal portion 240, a distal portion 230, and a connector portion 206 disposed between the proximal portion 240 and the distal portion 230. As shown, the proximal portion 240 includes a hybrid cable comprising the optical cable 202 (e.g., fiber optics) and the detector cable 204 (e.g., USB cable). At the proximal end of the proximal portion 240, the optical cable 202 includes an optical connector 208 configured to be connected to an illumination source and the detector cable 210 includes the detector connector 210 configured to be connected to a computing device. The connector of the optical cable may be sealed in some embodiments. Correspondingly, the detector cable may have a detachable cap 216 associated with the detector connector 210 to seal the interior of the detector cable. Alternatively, a sealed detector cable may be used. In some instances, during sterilization of the imaging device, the detector cap 216 may be capped onto the detector connector 210 to prevent exposure of the electronics within the detector cable to sterilization gases in the surrounding environment (e.g., H₂O₂ plasma).

As shown in FIG. 5A, the optical cable 202 and the detector cable 204 may be integrated into a single cable bundle 201 after passing through the yoke 206. The yoke 206 may function to combine the separate proximal portions of the different cables into a single monolithic cable bundle 201 extending distally from the yoke. In some instances, a cable sheath may be employed to bundle the hybrid cables into the single cable bundle 201 in the distal portion 230 of the cable assembly 190. In some embodiments, the yoke and the cable sheath may be sealed relative to the exterior environment. Additionally, the proximal portions of the optical cable and detector cable may form a seal with the yoke such that the overall hybrid cable assembly may be sealed. At the distal portion 230, the monolithic cable bundle 201 (including the optical cable 202 and the detector cable 204) may be passed through the tapered housing portion 150. The distal end 120 of the optical cable 202 may extend into the housing 116 of the body 112 (e.g., as shown in FIG. 2) and function as the light source for providing light into the body of the device. The distal end of the detector cable 204 within the cable bundle 201 may pass through and exit the tapered housing portion 150 as a data output connector 123. The data output connector 123 may be connected to the data output 122 of the photosensitive detector 118 (as shown in FIG. 2). According to the embodiments shown in FIG. 5A, the cable assembly further comprises a pressure unit associated with (e.g., integrated into) a portion of the cable assembly. The pressure unit may comprise a pressure inlet 250 which may be coupled to, or otherwise integrated with, the yoke 218 of the cable assembly 206. The pressure inlet may be in fluid communication with an interior volume of the housing of the device through the cable bundle. For example, either an open volume of the cable volume may provide fluid communication between the housing interior volume and pressure inlet and/or a separate pressure conduit may extend between the housing interior volume and pressure inlet through the cable bundle.

FIGS. 5B-5E depict various schematic illustrations of a portion (e.g., the yoke 206) of the cable assembly 190 of FIG. 2. FIG. 5B depicts a top view of the yoke 206. As shown, a pressure unit 218 comprises a pressure inlet 250 associated with the yoke 250 and a pressure conduit (not shown) extending into the distal portion 230 of the cable assembly 190 corresponding to the sealed cable bundle. As shown, the pressure inlet may have a removable plug 252 that can be used seal the pressure inlet 250. For example, during sterilization, the removable plug 252 may be used to seal the pressure inlet to block fluidic communication of the interior of the housing with the surrounding environment. However, permanent seals may also be used after a manufacturing process has been completed as well as the disclosure is not so limited.

FIG. 5C depicts a cross-sectional view of the cable bundle 201 in the distal portion 230 of the cable assembly 190 of FIG. 5B taken along 5C-5C at a position distal from the yoke. As shown, the cable bundle 201 comprises the pressure conduit 219, the detector cable 204 (e.g., USB cable), and the optical cable 202 (e.g., fiber optics) encapsulated, or otherwise surrounded and sealed, by a cable sheath 225 to form a sealed interior volume that is isolated from the external atmosphere surrounding the cable assembly. The inner diameter of the cable sheath 225 is sized such that the interior components fit into the sheath inner volume. This may either be a loose or tight fitting of the components within the sheath interior depending on the particular embodiment.

FIGS. 5D-5E depict various cross-sectional views of the yoke 206 of FIG. 2 taken along 5D-5D and 5E-5E. As shown, the pressure conduit 219 may extend from the pressure inlet 250 through the interior of the yoke 206 to the cable bundle 201, together with the hybrid cable (optical cable 202 and detector cable 204). The pressure conduit 219 may be integrated into and extend along a length of the cable bundle 201 along with the hybrid cable such that the overall cable assembly extends into an interior volume of the housing of the device (e.g., housing 116 as shown in FIG. 2). As shown in FIG. 2, the pressure inlet 250 may be in fluidic communication with an interior volume of the housing 116 through the cable assembly 190.

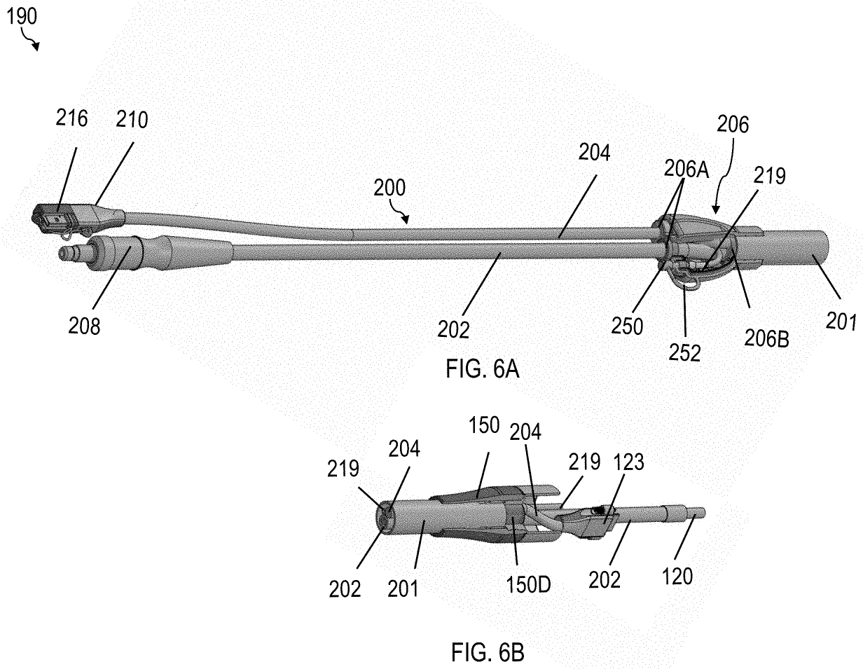

FIGS. 6A-6B depict perspective views of various portions of the cable assembly 190 of FIG. 2. FIG. 6A depicts a perspective view of the hybrid cable 200 and the yoke 206 of the cable assembly 190. FIG. 6B depicts a perspective view of the tapered housing portion 150 that extends distally from the yoke 206 of FIG. 6A. As shown in FIG. 6A, the cable assembly comprises the yoke 206 configured to receive the hybrid cable 200 (optical cable 202 and detector cable 204) via various connector openings 206A that may be sized and shaped to accept the different cables. These openings may form a slip fit or compression fit with the cables during assembly. Additionally, a sealant, such as one or more adhesive sealants, may be used to seal the cables within the corresponding openings. The pressure inlet 250 may be incorporated into the yoke 206 and the pressure conduit 219 may be fluidly connected to and extend from the pressure inlet 250 into the yoke 206. The pressure conduit 219 and hybrid cable (202 and 204) may exit the yoke 206 as the monolithic cable bundle 201, as described with respect to FIGS. 5A-5E. The cable bundle 201 may be sealed to a distal portion of the yoke, as shown in FIG. 6B, may then extend into the tapered housing 150 and further extend into an interior of the housing of the imaging device (housing 116 of device body 112 in FIG. 2). In some embodiments, a seal plug may be formed using an appropriate structural adhesive disposed between an exterior portion of a sheath of the cable bundle 201 and an interior surface of the housing to form a sealed pass through into an interior volume of the housing. Accordingly, as shown in FIG. 6B, the pressure conduit 219 exiting the tapered housing 150 may be in fluidic communication with the interior of the housing of the device while also providing a sealed construction for the hybrid cable and housing. Other components shown in FIGS. 6A-6B (e.g., detector cap 216, detector connector 210, optical connector 208, data output connector 123, light source 120, etc.) have already been described in detail in FIG. 5A. While a pressure inlet associated with the yoke has been described above, it should be understood that the pressure inlet may be associated with any appropriate portion of the cable assembly, imaging device housing, and/or any other appropriate portion of the overall imaging system as the disclosure is not limited in this fashion.

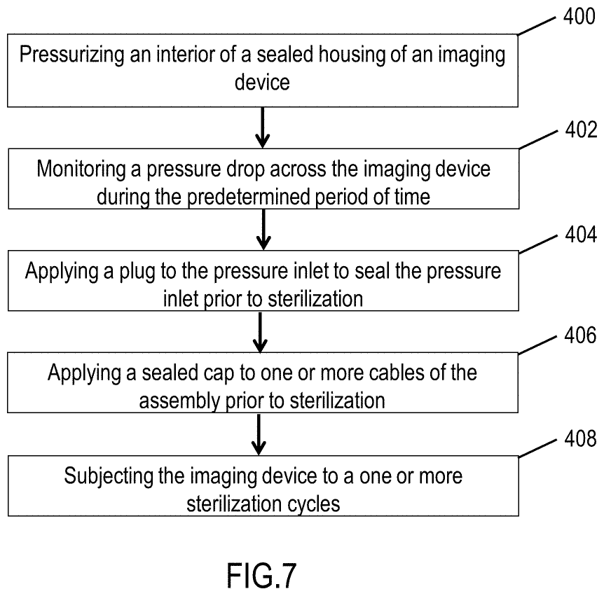

FIG. 7 depicts a flow chart of a method of manufacturing the imaging device of FIG. 2. After assembling the imaging device, a pressure test may be performed to check whether the device has been properly sealed. To conduct the pressure test, a positive pressure is first introduced via the pressure inlet into the interior of a sealed housing of the imaging device (e.g., step 400 in FIG. 7). For example, as shown in FIG. 2, a positive pressure may be applied via the pressure inlet 250 to pressurize an interior of the sealed housing 116 in FIG. 2. Next, a pressure drop within the sealed housing of the imaging device may be monitored over a predetermined period of time (e.g., step 402 in FIG. 7). If the monitored pressure drop is within a predetermined value (e.g., less than or equal to 10 kPa, less than or equal to 5 kPa, or other pressure drop described herein), the imaging device has been properly sealed and may be subjected to sterilization cycles. Prior to sterilizing the imaging device, the various openings and inlets of the imaging device may be properly sealed. As shown in FIG. 2, the detector connector 210 (e.g., USB connector) may be sealed with the removable detector cap 216 (e.g., step 406 in FIG. 7). Similarly, the pressure inlet 250 in FIG. 2 may be sealed with the removable pressure plug 216, as shown in FIGS. 5A-6A (e.g., step 404 in FIG. 7), or a permeant seal may be applied in some embodiments. Upon sealing the various opening and/or inlets, the imaging device may be subjected to a number of sterilization cycles both prior to an initial use as well as after subsequent uses of the imaging device (e.g., step 408 in FIG. 7). During sterilization, the device may be exposed to a sterilization gas (e.g., H₂O₂ plasma). The imaging device may be capable of withstanding a relatively high number of sterilization cycles, as described elsewhere herein.

Turning again to FIG. 3, the figure depicts a partially exploded view of one embodiment of a probe of a sterilizable handheld medical imaging device comprising various light absorbing and/or gas-tight features and/or constructions, according to some embodiments. For example, at least a portion of the interior surfaces of the imaging device may comprise anodized aluminum. Example of interior surfaces that may be anodized include an interior surface of the housing 116, an interior surface of the rigid imaging tip 102, an interior surface 114B of the light covering portion 114, an interior surface 150B of the tapered housing portion 150, and/or other surfaces which may be disposed along an optical path extending through the device and/or may otherwise be subjected to incident stray light. In some cases, the anodized interior surfaces of component 116, 114, 102, and 150 may be capable of at least partially absorbing any light that deviates from an optical path (e.g., the illumination path 139 and the imaging path 140 as shown in FIGS. 4A-4B). As shown in FIG. 4A-4B, the illumination path 139 comprises a path along which light travels from an illumination source to the distal portion of the rigid imaging tip via the mirror 129, the dichroic mirror 124, and the mirror 123. An imaging path comprises a path along which light travels from the distal portion 104 of the rigid imaging tip 102 to the photosensitive detector 118 via the dichroic mirror 124. It should be noted that any non-optical interior surfaces within the device may be anodized. Examples of interior surfaces that may not be anodized include the light source mirror 129, the mirror 123, the dichroic mirror 124, the aperture stop 132, the lens 126, the filters 130, the photosensitive detector 118, the transparent windows 105, and other optical components of an imaging device. In some instances, the optical components and associated surfaces described above (e.g., mirrors, lens, filters, windows, etc.) comprise a corrosion resistant material. In some cases, the corrosion resistant material may be a material that is resistant to the sterilization gases (e.g., H₂O₂ plasma). Non-limiting examples of such materials include various types of glass, quartz, polymer optics, and/or metals comprising corrosion-resistant coatings (e.g., MgF₂). For example, in one embodiment, one or more of the mirrors may comprise a metal (e.g., aluminum, gold, etc.) coated with a corrosion resistant material (e.g., MgF₂). In some embodiments, the optical components and associated surfaces described above may comprise glass, quartz, and/or polymer optics, etc.

In some embodiments, one or more of the mirrors (e.g., the mirror 123, the mirror 129, etc.) may have an exterior surface that is exposed to a surrounding environment. For example, as shown in FIG. 3, the exterior surface of the one or more mirrors (e.g., the mirror 123, the mirror 129, etc.) may form a portion of the exterior surface of the housing 116 and/or the rigid imaging tip 102. In some such embodiments, the exterior surface of the mirrors comprises a material (e.g., a biocompatible anodized aluminum, stainless steel, etc.) resistant to sterilization. Additionally, the one or more mirrors may have an interior surface (e.g., a surface facing the interior of the housing 116 or rigid imaging tip 102) comprising a corrosion resistant material described above (e.g., metals coated with MgF₂). In another embodiment, the one or more mirrors (e.g., the mirror 123, the mirror 129, etc.) may be entirely sealed within the housing and/or the rigid imaging tip. That is, both surfaces of the one or more mirrors may be positioned within an interior of the housing and/or the rigid imaging tip. In some such embodiments, both surfaces of the one or more mirrors may comprise a corrosion resistant material and/or coating described above (e.g., glass coated with aluminum, metals coated with MgF₂, etc.).

In accordance with some embodiments as shown in FIG. 3, the imaging probe 100 may comprise a plurality of anodized exterior surfaces that are resistant to sterilization gases (e.g., H₂O₂ plasma). The anodized exterior surfaces may comprise biocompatible anodized aluminum, which in some instances may also be applied to the interior surfaces of these components as well. Other biocompatible anodized metals as well as other biocompatible coatings, which may also be light absorbing, may also be used as the present disclosure is not so limited. Non-limiting examples of anodized exterior surfaces include an exterior surface 114A of the light covering portion 114, an exterior surface 150A of the tapered housing portion 150, an exterior surface of the housing 116, an exterior surface 102A of the imaging tip 102, and/or an exterior surface of the removable tip 103, etc. It should be noted that any exterior surfaces associated with the housing enclosing the imaging tip and body of the device may be anodized, as the present disclosure is not so limited. In some cases, the exterior surfaces associated with the cable assembly extending out from the housing 116 may also be sterilizable. As shown in FIG. 2, the exterior surfaces associated with cable assembly 190, such as a sheath, yoke, or other portion of the cable assembly, may either be made from or be coated with a material that is resistant to corrosion, embrittlement, or other degradation by the sterilization gases. This may include materials such as a thermoplastic vulcanizate, a silicone, a thermoplastic natural rubber (TPNR), a thermoplastic epoxidized natural rubber (TPENR) and/or any other appropriate materials as described above.

As shown in FIG. 2 and FIG. 3, the imaging device 100 may be assembled from individual pieces, such as the rigid imaging tip 102, the various housing and coverings (e.g., 114, 116, 150), the sealed cable assembly 119, etc. To form a properly sealed imaging device capable of withstanding sterilization cycles, various types of sealed joints, seams, pass throughs, and other structures may be employed. For example, as shown in FIG. 3, various lap joints may be employed for creating seals between various components. As an example, the light covering portion 114 may be sealed to the housing 116 via sealed lap joints. To do so, an edge 116A of the housing 116 may be shaped to form a lap joint, or other appropriate joint, with an edge 114C of the light covering portion 114 (which may be viewed as a portion of the overall housing) to form a joint. Similarly, an edge 114D of the light covering portion 114 may be joined with an edge 150C of the tapered housing portion 150 to form a lap joint or other appropriate joint. The joints may be sealed using either a single or multiple sealing adhesives and/or materials. For example, a first sealant may be used on an interior portion of the various illustrated joints and a second sealant may be placed on an exterior seam of the different joints. An exemplary sealed joint is discussed further below according to the embodiment illustrated in FIG. 9. Other structures that may be sealed using overlapping joints and sealing adhesives in the overall imaging device may include, but are not limited to, a seal between the imaging tip 102 and the housing 116, the mirror 123 and the imaging tip 102, the light source mirror 129 and the housing 116, and/or any other appropriate combination of surfaces as the disclosure is not so limited. The cable assembly may be sealed with the housing as previously described. Additional components, such as a window on a distal end portion of the rigid imaging tip 102 may be sealed using any appropriate combination of gaskets, sealing adhesives, overlapping portions of the window and a supporting ledge on a distal portion of the imaging tip, and/or any other appropriate sealing method. While the use of lap joints is primarily discussed above, it should be understood that any appropriate joint capable of being sealed may be used in the various embodiments disclosed herein. Additionally, a number of the components described above may be combined into an integrally formed structure such that separate seals may not be needed in all of the locations described herein as different constructions of an imaging device exterior are contemplated.

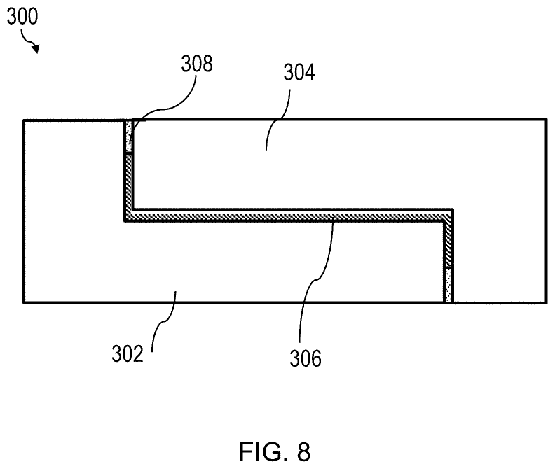

FIG. 8 depicts a schematic side view of a sealed lap joint 300, according to some embodiments. To form the sealed lap joint, a first edge 302 and a second corresponding edge 304 of two separate structures may be first overlapped to form a joint. To form the first seal, a first sealing adhesive 306 (e.g., a structural adhesive such as epoxy) may be applied to an exterior surface along the perimeter of the joint formed from the first edge 302 and the second corresponding edge 304 as noted above. The first sealing adhesive may be applied along one or both external perimeters of the joint. Upon curing of the first sealing adhesive, a second sealing adhesive 308 (e.g., a UV curable material) may be applied to an exterior surface, or an interior portion of the joint adjacent to the exterior surface, along the external perimeter of the joint. This may either overcoat and/or fill the external perimeter of the joint 300. Such a double sealed connection may be applied to any of the sealed joints described herein.

In some embodiments, the imaging device comprises various sealed pass throughs. As shown in FIG. 3, the imaging device 100 includes the tapered housing portion 150 that may be configured to pass the cable(s) (e.g., the cable bundle 201) into an interior of the housing 116 of the device. To help form a seal, a structural adhesive (e.g., epoxy) may be used to seal the pass through between tapered housing portion 150 with the cable(s). For example, as shown in FIG. 6B, the tapered portion 150 and the cable bundle 201 may be sealed by a structural adhesive 150D. Various other pass throughs present in the imaging device 100 may be sealed in a similar fashion. For example, as shown in FIG. 6A, the pass through between the yoke 206 and the hybrid cable 200 may be sealed via a structural adhesive 206B.

The sealed imaging device described with respect to FIG. 3 may comprise various other gas-tight or waterproof constructions. For example, the exterior 102A of the imaging tip 102 may be fabricated from a single piece of metal (e.g., aluminum). This may reduce the number of joints present in the imaging tip and reduces the chance for leakage. As another example, the various mirrors (e.g., the mirror 123 disposed at the junction between the proximal portion and the distal portion of the rigid imaging tip and/or the mirror 129) may be glued, sealed, or otherwise connected onto the imaging tip in a gas tight manner during device manufacturing using appropriately sealed joints as described herein.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A sterilizable handheld medical imaging device, comprising:
a housing, wherein an interior of the housing is sealed from a surrounding environment;
a photosensitive detector disposed in the housing;
a rigid imaging tip extending distally from the housing and optically coupled with the photosensitive detector; and
a sealed cable assembly extending out from the housing, wherein the cable assembly is adapted and arranged to be selectively connected to an illumination source and wherein the cable assembly is configured be selectively connected to a computing device.

2. The device of claim 1, wherein the imaging device is a fluorescence imaging device.

3. The device of claim 1, wherein the imaging device is configured to be sterilized with a H₂O₂ plasma.

4. The device of claim 1, wherein the imaging device is capable of withstanding at least 10 sterilization cycles.

5. The device of claim 1, wherein at least a portion of external surfaces of the imaging device comprises a coating resistant to sterilization.

6. The device of claim 5, wherein at least a portion of the external surfaces comprises biocompatible anodized aluminum.

7. The device of claim 1, wherein at least a portion of interior surfaces of the imaging device comprises biocompatible anodized aluminum.

8. The device of claim 6, wherein the anodized surfaces are light absorbing.

9. The device of claim 6, wherein the anodized surfaces are black.

10. The device of claim 1, wherein an exterior of the sealed cable assembly comprises thermoplastic vulcanizate (TPV) and/or silicone.

11. The device of claim 1, wherein the sealed cable assembly comprises a hybrid cable comprising a fiber-optic cable and an electrical cable.

12. The device of claim 11, wherein the sealed cable assembly comprises a yoke adapted and arranged to bundle the hybrid cable into a monolithic cable bundle.

13. The device of claim 1, further comprising a pressure inlet in fluidic communication with an interior of the housing.

14. The device of claim 1, wherein one or more of the cables within the cable assembly comprises a removable cap configured to seal an end portion of the one or more cables during sterilization.

15. The device of claim 1, wherein the imaging device comprises sealed joints, seams, and/or pass throughs that are sealed with a structural adhesive and a light curable sealing material.

16. A sterilizable handheld medical imaging device, comprising:
a housing, wherein an interior of the housing is sealed from a surrounding environment;
a photosensitive detector disposed in the housing; and
a pressure inlet in fluidic communication with an interior of the housing.

17. The device of claim 16, further comprising a sealed cable assembly extending out from the housing, wherein the pressure inlet is in fluid communication with the interior of the housing through the cable assembly.

18. The device of claim 17, wherein the pressure inlet is integrated into the cable assembly via a yoke disposed at a junction between the proximal portion and the distal portion of the cable assembly.

19. The device of claim 17, wherein an exterior of the sealed cable assembly comprises thermoplastic vulcanizate (TPV) and/or silicone.

20. The device of claim 17, wherein the sealed cable assembly comprises a hybrid cable comprising a fiber-optic cable and an electrical cable.

21. The device of claim 17, wherein the sealed cable assembly comprises a yoke adapted and arranged to bundle a plurality of cables and the pressure inlet into a monolithic cable bundle.

22. The device of claim 16, wherein the pressure inlet is adapted and arranged to introduce a positive pressure into the imaging device.

23. The device of claim 16, further comprising a removable plug adapted and arranged to seal the pressure inlet.

24. The device of claim 16, further comprising a conduit arranged and configured to extend from the pressure inlet into the interior of the housing.

25. The device of claim 16, wherein the imaging device is a fluorescence imaging device.

26. The device of claim 16, wherein the imaging device is configured to be sterilized with a H₂O₂ plasma.

27. The device of claim 16, wherein the imaging device is capable of withstanding at least 10 sterilization cycles.

28. The device of claim 16, wherein at least a portion of external surfaces of the imaging device comprises a coating resistant to sterilization.

29. The device of claim 28, wherein the at least a portion of the external surfaces comprises biocompatible anodized aluminum.

30. The device of claim 16, wherein at least a portion of interior surfaces of the imaging device comprises biocompatible anodized aluminum.

31. The device of claim 29, wherein the anodized surfaces are light absorbing.

32. (canceled)

33. A method of manufacturing an imaging device, the method comprising:
pressurizing an interior of a sealed housing of an imaging device; and
monitoring a pressure drop within the sealed housing of the imaging device over a predetermined period of time.

34. The method of claim 33, further comprising sealing the interior of the sealed housing.

35-39. (canceled)

40. The method of claim 38, wherein pressurizing an interior of the sealed housing comprises introduce a positive pressure into the sealed housing through the cable assembly.

41. The method of claim 33, wherein the pressure drop is less than or equal to 5 kPa psi over the predetermined period of time.

42. The method of claim 40, wherein the positive pressure comprises a pressure between 25 kPa and 40 kPa.

43. The method of claim 33, wherein the predetermined period of time is at least 5 minutes.

44. The method of claim 33, further comprising subjecting the imaging device to at least one sterilization cycle via exposure to a sterilization gas.

45. The method of claim 44, wherein the sterilization gas comprises H₂O₂ plasma.

46. The method of claim 44, further comprising applying a cap to seal an end portion of one or more cables in the cable assembly prior to subjecting the imaging device to the sterilization cycle.

47-55. (canceled)
